(19) United States
(12) Patent Application Publication
(10) Pub. No.: US 2025/0376657 A1
(43) Pub. Date: Dec. 11, 2025

(54) VISCOSITY-TOLERANT CORYNEBACTERIUM GLUTAMICUM STRAIN AND USE THEREOF

(71) Applicant: JIANGNAN UNIVERSITY, Wuxi (CN)

(72) Inventors: Zhen KANG, Wuxi (CN); Jian CHEN, Wuxi (CN); Litao HU, Wuxi (CN); Guocheng DU, Wuxi (CN); Ruirui XU, Wuxi (CN); Sen XIAO, Wuxi (CN)

(21) Appl. No.: 19/231,437

(22) Filed: Jun. 7, 2025

Related U.S. Application Data
(63) Continuation of application No. PCT/CN2024/104401, filed on Jul. 9, 2024.

(30) Foreign Application Priority Data
Jun. 6, 2024 (CN) 202410730365.3

(51) Int. Cl. C12N 1/20; C12N 9/04; C12N 9/10; C12N 9/90; C12P 19/04; C12R 1/15

(52) U.S. Cl. CPC C12N 1/205; C12N 9/0006; C12N 9/1051; C12N 9/1096; C12N 9/90; C12P 19/04; C12Y 101/01022; C12Y 204/01212; C12Y 206/01016; C12Y 504/02002; C12R 2001/15

(57) ABSTRACT

The Corynebacterium glutamicum strain of the present invention is obtained by mutating Corynebacterium glutamicum ATCC 13032, with the mutation sites including: mutating cytosine at site 862902 into thymine; mutating guanine at site 862903 into adenine; mutating cytosine at site 862953 into thymine; mutating adenine at site 862961 into guanine; inserting cytosine and thymine at site 862958; and mutating of guanine at site 862963 by deletion. The Corynebacterium glutamicum strain of the present invention exhibits significantly increased tolerance in high-viscosity environments and growth and metabolism ability under low dissolved oxygen conditions, thereby increasing the yield of mucopolysaccharides, and avoiding the problems where resulting mucopolysaccharides cause the fermentation broth to become viscous and have insufficient dissolved oxygen, which would further limit the metabolism of Corynebacterium glutamicum and ultimately affect the synthesis of mucopolysaccharides.

This application is a Continuation Application of PCT/CN2024/104401, filed on Jul. 9, 2024, which claims priority to Chinese Patent Application No. 202410730365.3, filed on Jun. 6, 2024, which is incorporated by reference for all purposes as if fully set forth herein.

A Sequence Listing XML file named “10015_0176.xml” created on Jun. 7, 2025 and having a size of 24,462 bytes, is filed concurrently with the specification. The sequence listing contained in the XML file is part of the specification and is herein incorporated by reference in its entirety.

### FIELD OF THE INVENTION

The present invention relates to the technical field of biological genetic engineering, in particular to a viscosity-tolerant Corynebacterium glutamicum strain and use thereof.

### DESCRIPTION OF THE RELATED ART

Corynebacterium glutamicum is a Gram-positive bacterium. Due to its properties such as clear genetic background, stable protein secretion, low extracellular hydrolase activity, and non-toxicity, it has now been widely applied in the production processes of various value-added chemicals, amino acids, and fuels. With the continuous revelation of gene regulation mechanism, designing and constructing Corynebacterium glutamicum using synthetic biology techniques for biomanufacturing has become a current research focus in this field. Moreover, Corynebacterium glutamicum is a strict aerobe, which requires the continuous introduction of sterile air during its fermentation process. Therefore, during the fermentation of Corynebacterium glutamicum to synthesize viscous substances, such as hyaluronic acid, chondroitin, heparin precursors, etc., or during the high-cell-density fermentation of Corynebacterium glutamicum, the accumulation of viscous substances and the increase in bacterial cell density will lead to a decrease in dissolved oxygen in the fermentation broth, thereby affecting its normal metabolic activities. This is also one of the current challenges in optimizing the fermentation process of Corynebacterium glutamicum. Hyaluronic acid (HA) is a type of linear acidic mucopolysaccharide, which has excellent moisturizing properties, is capable of playing effects such as inhibiting inflammation, and is widely used in the cosmetic and pharmaceutical fields. At present, most hyaluronic acid in the industrial market is obtained by fermentation of pathogenic microorganisms such as Streptococcus zooepidemicus and Escherichia coli K4. But its development in fields such as medicine is seriously restricted because of its pathogenic factors such as endotoxin. Using microorganisms with clear genetic background and high biological safety to synthesize hyaluronic acid, such as Corynebacterium glutamicum, has become the development trend of hyaluronic acid synthesis by microbial fermentation.

Although the yields of mucopolysaccharides such as hyaluronic acid, chondroitin, and heparin precursors synthesized by Corynebacterium glutamicum have been significantly increased at present, due to their super water-absorbing properties, the fermentation broth becomes viscous as the fermentation process progresses, thereby inhibiting the normal metabolic activities of cells and limiting further increase in the production of viscous substances such as hyaluronic acid, chondroitin, and heparin precursors. Therefore, it is expected to engineer Corynebacterium glutamicum by directional evolution to increase its tolerance in highly viscous solution, and then further increase the synthesis efficiency of mucopolysaccharides by Corynebacterium glutamicum.

### SUMMARY OF THE INVENTION

Therefore, the technical problem to be solved by the present invention is to overcome the problem that in the prior art, when Corynebacterium glutamicum is used to produce hyaluronic acid, the fermentation broth becomes viscous in the fermentation process, thereby reducing the oxygen content in the fermentation broth, further inhibiting the metabolic activities of Corynebacterium glutamicum, and finally affecting the yield of hyaluronic acid.

In order to solve the technical problem above, the present invention provides a Corynebacterium glutamicum strain, which is obtained by mutating Corynebacterium glutamicum ATCC 13032, with the mutation sites including: mutation of cytosine at site 862902 into thymine; mutation of guanine at site 862903 into adenine; mutation of cytosine at site 862953 into thymine; mutation of adenine at site 862961 into guanine; inserting cytosine and thymine at site 862958; and mutation of guanine at site 862963 by deletion. The Corynebacterium glutamicum strain of the present invention exhibits significantly increased tolerance in high-viscosity environments and significantly increased growth and metabolism ability under low dissolved oxygen conditions, thereby increasing the yield of mucopolysaccharides, and avoiding the problems where resulting mucopolysaccharides cause the fermentation broth to become viscous and have insufficient dissolved oxygen, which would further limit the metabolism of Corynebacterium glutamicum and ultimately affect the synthesis of mucopolysaccharides.

The first object of the present invention is to provide a viscosity-tolerant Corynebacterium glutamicum strain, the Corynebacterium glutamicum strain is obtained by mutating Corynebacterium glutamicum ATCC 13032, with mutation sites including:
(1) mutating cytosine at site 862902 into thymine;
(2) mutating guanine at site 862903 into adenine;
(3) mutating cytosine at site 862953 into thymine;
(4) mutating adenine at site 862961 into guanine;
(5) inserting cytosine and thymine at site 862958; and
(6) mutating of guanine at site 862963 by deletion.

Preferably, the mutation sites include:

| type of gene | genome start | genome termination | reference | CG-HAT |
|---|---|---|---|---|
| mutation | position | position | genome | genome |
| single nucleotide | 76620 | 76620 | G | A |
| variation | | | | |
| single nucleotide | 143559 | 143559 | G | A |
| variation | | | | |
| single nucleotide | 245282 | 245282 | C | A |
| variation | | | | |
| single nucleotide | 286678 | 286678 | G | A |
| variation | | | | |
| single nucleotide | 287836 | 287836 | T | C |
| variation | | | | |
| single nucleotide | 349179 | 349179 | G | A |
| variation | | | | |
| single nucleotide | 547272 | 547272 | G | C |
| variation | | | | |
| single nucleotide | 591573 | 591573 | A | G |
| variation | | | | |
| single nucleotide | 645542 | 645542 | G | A |
| variation | | | | |
| single nucleotide | 661760 | 661760 | T | C |
| variation | | | | |
| single nucleotide | 862902 | 862902 | C | T |
| variation | | | | |
| single nucleotide | 862903 | 862903 | G | A |
| variation | | | | |
| single nucleotide | 862953 | 862953 | C | T |
| variation | | | | |
| single nucleotide | 862961 | 862961 | A | G |
| variation | | | | |
| single nucleotide | 878149 | 878149 | G | A |
| variation | | | | |
| single nucleotide | 933781 | 933781 | A | T |
| variation | | | | |
| single nucleotide | 1136848 | 1136848 | G | T |
| variation | | | | |
| single nucleotide | 1162092 | 1162092 | T | G |
| variation | | | | |
| single nucleotide | 1480319 | 1480319 | A | G |
| variation | | | | |
| single nucleotide | 1589428 | 1589428 | T | C |
| variation | | | | |
| single nucleotide | 1890744 | 1890744 | C | A |
| variation | | | | |
| single nucleotide | 1961649 | 1961649 | C | A |
| variation | | | | |
| single nucleotide | 1961652 | 1961652 | C | T |
| variation | | | | |
| single nucleotide | 1961654 | 1961654 | C | T |
| variation | | | | |
| single nucleotide | 1961657 | 1961657 | T | C |
| variation | | | | |
| single nucleotide | 1961768 | 1961768 | A | T |
| variation | | | | |
| single nucleotide | 1961774 | 1961774 | C | T |
| variation | | | | |
| single nucleotide | 1961785 | 1961785 | T | C |
| variation | | | | |
| single nucleotide | 1961792 | 1961792 | A | T |
| variation | | | | |
| single nucleotide | 1961798 | 1961798 | C | A |
| variation | | | | |
| single nucleotide | 1961807 | 1961807 | G | A |
| variation | | | | |
| single nucleotide | 1961810 | 1961810 | T | A |
| variation | | | | |
| single nucleotide | 1961820 | 1961820 | C | T |
| variation | | | | |
| single nucleotide | 1961836 | 1961836 | C | T |
| variation | | | | |
| single nucleotide | 1962180 | 1962180 | T | A |
| variation | | | | |
| single nucleotide | 1963988 | 1963988 | G | A |
| variation | | | | |
| single nucleotide | 1963993 | 1963993 | T | C |
| variation | | | | |
| single nucleotide | 1964078 | 1964078 | A | C |
| variation | | | | |
| single nucleotide | 1964435 | 1964435 | G | C |
| variation | | | | |
| single nucleotide | 1965170 | 1965170 | A | G |
| variation | | | | |
| single nucleotide | 1965284 | 1965284 | C | T |
| variation | | | | |
| single nucleotide | 1965286 | 1965286 | T | G |
| variation | | | | |
| single nucleotide | 1965326 | 1965326 | A | G |
| variation | | | | |
| single nucleotide | 1965341 | 1965341 | A | T |
| variation | | | | |
| single nucleotide | 1965344 | 1965344 | C | A |
| variation | | | | |
| single nucleotide | 1965349 | 1965349 | C | A |
| variation | | | | |
| single nucleotide | 1965437 | 1965437 | C | G |
| variation | | | | |
| single nucleotide | 1965443 | 1965443 | A | G |
| variation | | | | |
| single nucleotide | 1965445 | 1965445 | C | T |
| variation | | | | |
| single nucleotide | 1965455 | 1965455 | C | T |
| variation | | | | |
| single nucleotide | 1965500 | 1965500 | A | G |
| variation | | | | |
| single nucleotide | 1965503 | 1965503 | T | G |
| variation | | | | |
| single nucleotide | 1965505 | 1965505 | C | T |
| variation | | | | |
| single nucleotide | 1965506 | 1965506 | A | T |
| variation | | | | |
| single nucleotide | 1965514 | 1965514 | A | G |
| variation | | | | |
| single nucleotide | 1965677 | 1965677 | T | G |
| variation | | | | |
| single nucleotide | 2036533 | 2036533 | T | C |
| variation | | | | |
| single nucleotide | 2131457 | 2131457 | A | C |
| variation | | | | |
| single nucleotide | 2391582 | 2391582 | C | T |
| variation | | | | |
| single nucleotide | 2499503 | 2499503 | A | G |
| variation | | | | |
| single nucleotide | 2578510 | 2578510 | T | C |
| variation | | | | |
| single nucleotide | 2587299 | 2587299 | T | G |
| variation | | | | |
| single nucleotide | 2643621 | 2643621 | C | T |
| variation | | | | |
| single nucleotide | 2676334 | 2676334 | G | A |
| variation | | | | |
| single nucleotide | 2676510 | 2676510 | A | G |
| variation | | | | |
| single nucleotide | 2693877 | 2693877 | A | T |
| variation | | | | |
| single nucleotide | 2701401 | 2701401 | T | C |
| variation | | | | |
| single nucleotide | 2749153 | 2749153 | C | A |
| variation | | | | |
| single nucleotide | 2762168 | 2762168 | G | A |
| variation | | | | |
| single nucleotide | 2965869 | 2965869 | C | T |
| variation | | | | |
| single nucleotide | 3026302 | 3026302 | G | C |
| variation | | | | |
| single nucleotide | 3026303 | 3026303 | C | A |
| variation | | | | |
| single nucleotide | 3114280 | 3114280 | G | C |
| variation | | | | |
| single nucleotide | 3118769 | 3118769 | A | G |
| variation | | | | |
| single nucleotide | 3136899 | 3136899 | A | G |
| variation | | | | |
| single nucleotide | 3217551 | 3217551 | A | T |
| variation | | | | |
| single nucleotide | 3272034 | 3272034 | G | A |
| variation | | | | |
| Insertion | 76636 | 76636 | — | T |
| Insertion | 862572 | 862572 | — | T |
| Insertion | 862958 | 862958 | — | CT |
| Deletion | 862963 | 862963 | G | — |
| Deletion | 1149598 | 1149599 | TC | — |
| Insertion | 1904543 | 1904543 | — | T |
| Deletion | 2312377 | 2312427 | CGGTG | T |
| | | | TCATCC | |
| | | | TAGAG | |
| | | | ATTAAG | |
| | | | GCTGA | |
| | | | AGAGG | |
| | | | ATGAC | |
| | | | ACCGT | |
| | | | CGACG | |
| | | | TCGG | |
| Insertion | 2774003 | 2774003 | — | GC |
| Deletion | 2774005 | 2774006 | GA | — |

The second object of the present invention is to provide use of the Corynebacterium glutamicum strain above in production of mucopolysaccharides.

Preferably, the mucopolysaccharide includes hyaluronic acid.

The third object of the present invention is to provide recombinant Corynebacterium glutamicum strain, the modification of the recombinant Corynebacterium glutamicum strain includes: overexpressing a hyaluronic acid synthase, and overexpressing at least one of a glutamine-fructose-6-phosphate aminotransferase, a phosphoglucomutase, and a uridine diphosphate-glucose dehydrogenase.

Preferably, the gene sequence of the glutamine-fructose-6-phosphate aminotransferase (glmS) is as shown in SEQ ID NO. 17, the gene sequence of the phosphoglucomutase (glmM) is as shown in SEQ ID NO. 18, and the gene sequence of the uridine diphosphate-glucose dehydrogenase (ugd) is as shown in SEQ ID NO. 19. Hyaluronic acid is mainly synthesized through the UDP-N-acetylglucosamine pathway and the UDP-glucuronic acid pathway. The enzymes expressed by the ugd, glmS, and glmM genes are key enzymes in these two pathways, and enhancing the synthesis of the three enzymes can effectively increase the yield of hyaluronic acid.

Preferably, the overexpression is initiated by the Ptac promoter or the Ptrc promoter. The ugd, glmS, and glmM genes themselves also possess promoters. However, the promoter sequences of the ugd, glmS, and glmM genes are not clearly defined, and their expression capabilities are weak. The Ptac promoter and the Ptrc promoter are recognized as strong promoters with high expression capabilities. Therefore, by introducing the Ptac promoter or the Ptrc promoter in front of the ugd, glmS, and glmM genes, the expression of ugd, glmS, and glmM can be enhanced, thereby increasing the yield of hyaluronic acid.

The sixth object of the present invention is to provide a method for producing mucopolysaccharides, including a step of fermentation of the recombinant Corynebacterium glutamicum strain above.

Preferably, the fermentation step include: inoculating the recombinant Corynebacterium glutamicum strain into a fermentation medium for culture, adding IPTG to induce gene expression, centrifuging the fermentation broth after fermentation, and collecting the supernatant to obtain the mucopolysaccharide.

Preferably, the temperature of the fermentation process is 15-40° C. Temperature will affect the growth of the Corynebacterium glutamicum strain, which can grow at 15-40° C., and the optimum growth temperature of the Corynebacterium glutamicum strain is 30° C. Preferably, the temperature of the fermentation process is 30° C.

Further, the pH of the fermentation process is 6.5-7. pH will affect the growth of the Corynebacterium glutamicum strain, which can grow at pH 5-9, and the optimum pH of the Corynebacterium glutamicum strain is 7. Preferably, the pH of the fermentation process is 6.5-7.

Beneficial effects of the present invention:

The Corynebacterium glutamicum strain of the present invention exhibits significantly increased tolerance in high-viscosity environments and significantly increased growth and metabolism ability under low dissolved oxygen conditions, thereby increasing the yield of mucopolysaccharides (e.g., hyaluronic acid), and avoiding the problems where resulting mucopolysaccharides cause the fermentation broth to become viscous and have insufficient dissolved oxygen, which would further limit the metabolism of Corynebacterium glutamicum and ultimately affect the synthesis of mucopolysaccharides.

### BRIEF DESCRIPTION OF THE DRAWINGS

In order to make the contents of the present invention more clearly understood, the present invention will be further described in detail according to specific examples of the present invention and with the accompanying drawings, in which

FIG. 1 is a directed evolution screening process of the viscosity-tolerant Corynebacterium glutamicum strain;

FIG. 2 is change curves of OD₆₀₀ and glucose consumption of the viscosity-tolerant Corynebacterium glutamicum strain under different concentrations of hyaluronic acid;

FIG. 3 is the yield of hyaluronic acid obtained by fermentation of the viscosity-tolerant Corynebacterium glutamicum strain in a 5 L fermenter; and

FIG. 4 is a histogram of the yield of hyaluronic acid obtained by expressing different genes respectively in the hyaluronic acid synthesis pathways.

### DETAILED DESCRIPTION OF THE PREFERRED EMBODIMENTS

The present invention will be further described with the attached drawings and specific examples, so that those skilled in the art can better understand and implement the present invention, but the examples given are not taken as limitations of the present invention.

Strain: a wild-type Corynebacterium glutamicum strain (Corynebacterium glutamicum ATCC 13032);
Plasmid: pXMJ19, and pk18mobsacb;
LB medium: yeast powder 5 g/L, peptone 10 g/L and sodium chloride 10 g/L; BHI: brain heart infusion 37 g/L, and sorbitol 91 g/L;
Fermentation medium: glucose 40 g/L, corn steep liquor powder 20 g/L, (NH₄)₂SO₄ 30 g/L, KH₂PO₄ 1 g/L, K₂HPO₄ 1 g/L, MgSO₄ 25 g/L, and 3-(N-morpholino)propanesulfonic acid (MOPS) 42 g/L.

Preparation of competent cells of Corynebacterium glutamicum: A single colony from a plate was picked, inoculated into a shaking tube with 5 mL of the BHI liquid medium, and cultured overnight at 30° C. It was inoculated into a baffled Erlenmeyer flask with 50 mL of BHI at an inoculation amount of initial OD₆₀₀=0.2, and cultured at 30° C. until OD₆₀₀=1.4-1.6. The seed liquid was collected in a centrifuge tube, left in an ice bath for 10 min, and centrifuged at 4° C. for 5 min at 4000 rpm to collect the bacterial cells. The bacterial cells were resuspended with 20 mL of a 10% glycerol solution, and centrifuged at 4° C. for 5 min at 4000 rpm to collect the bacterial cells. The aforementioned operation was repeated twice. The bacterial cells were resuspended with 2.5 mL of a 10% glycerol solution, aliquoted into pre-chilled sterile EP tubes, and stored at −80° C. for use in electroshock transformation.

Purification of hyaluronic acid: The fermentation broth was collected and centrifuged at 10000 rpm for 5 min. An appropriate amount of supernatant was taken, into which 4 volumes of absolute ethanol were added, left in a 4° C. environment overnight for alcohol precipitation, and centrifuged at 10000 rpm for 5 min to discard the supernatant. After ethanol evaporation, the precipitate was resuspended in the original volume of water, fully dissolved, and centrifuged at 10000 rpm for 10 min to collect the supernatant. The aforementioned operation was repeated. After secondary alcohol precipitation, the supernatant was collected as the purified hyaluronic acid sample.

Determination of yield of hyaluronic acid: Sodium tetraborate decahydrate (4.77 g) was weighed and dissolved in 500 mL of concentrated sulfuric acid to prepare a solution of borax in sulfuric acid; 1.25 g of carbazole was weighed and dissolved in 500 mL of absolute ethanol to prepare a carbazole solution; and a 1 g/L glucuronic acid solution was prepared.

The purified hyaluronic acid sample was taken, and diluted by 10 to 100 times, 200 μL of which was pipetted into a glass colorimetric tube. Meanwhile, 1 mL of the solution of borax in sulfuric acid was added. The mixture was mixed well, then left in a boiling water bath for 15 min, and cooled on ice. 50 μL of the carbazole solution was added. The mixture was mixed well, and then left in a boiling water bath for 10 min. 200 μL of the reaction solution was added into a 96-well transparent plate, and determined the absorbance of the sample at a wavelength of 530 nm using a microplate reader. The 1 g/L glucuronic acid solution was gradient-diluted to 0, 10, 20, 30, 40, and 50 mg/L. After the borax in sulfuric acid-carbazole colorimetric reaction, the absorbance was determined at 530 nm. Using the absorbances as the abscissa and the glucuronic acid concentrations (mg/L) as the ordinate, a relation curve between the absorbance and the glucuronic acid concentration was plotted, thereby obtaining a standard curve equation: y=121.7x−6.035, with R² of 0.999.

The determined absorbance of the sample mentioned above was substituted into the standard equation to calculate the content of hyaluronic acid in the sample. Formula for calculating content of hyaluronic acid:

$$content of hyaluronic acid ( g / L ) = ( concentration determined from standard curve * dilution factor * 2.067 ) / 1000.$$

### Example 1: Screening of Viscosity-Tolerant Corynebacterium glutamicum Strain

(1) Screening of Corynebacterium glutamicum Strain CG-HAT

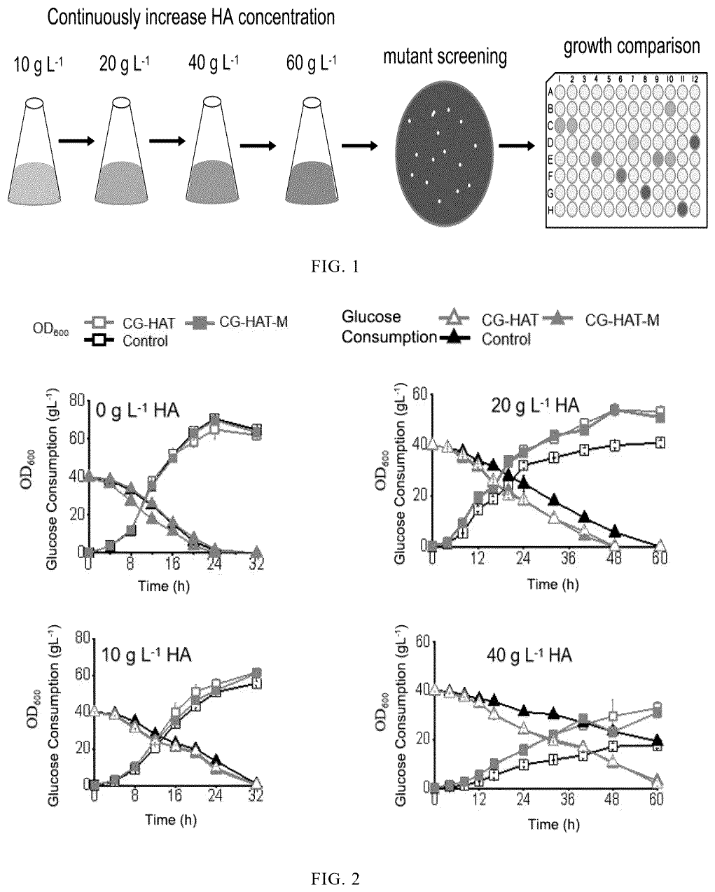

A single colony of Corynebacterium glutamicum ATCC 13032 was picked, inoculated into 5 mL of the BHI medium, and cultured overnight at 30° C. It was inoculated into 25 mL of the fermentation medium at an inoculation amount of initial OD₆₀₀=0.2, and allowed to grow until it reached the stationary phase. Then, it was inoculated into 25 mL of the fermentation medium containing 10 g/L HA at an inoculation amount of initial OD₆₀₀=0.2. After 20 h of fermentation, 2 M NaOH was added to adjust the pH of the fermentation broth to neutral. After the strain was fermented until it reached the stationary phase, it was inoculated into 25 mL of the fermentation medium containing 20 g/L HA at an inoculation amount of initial OD₆₀₀=0.2. After 20 h of fermentation, 2 M NaOH was added to adjust the pH of the fermentation broth to neutral. After the strain was fermented until it reached the stationary phase, it was inoculated into 25 mL of the fermentation medium containing 40 g/L HA at an inoculation amount of initial OD₆₀₀=0.2. After 20 h of fermentation, 2 M NaOH was added to adjust the pH of the fermentation broth to neutral. After the strain was fermented until it reached the stationary phase, it was inoculated into 25 mL of the fermentation medium containing 60 g/L HA at an inoculation amount of initial OD₆₀₀=0.2. After 20 h of fermentation, 2 M NaOH was added to adjust the pH of the fermentation broth to neutral. As the strain was cultured in the viscous fermentation broth, the HA content in the medium was gradually increased (as shown in FIG. 1).

During the subculture process, the strains in the fermentation broth were periodically streaked for isolation, and randomly picked single colonies were subjected to directed evolution effect verification. Using wild-type Corynebacterium glutamicum ATCC 13032 as the control, mutant strains were obtained by screening the strain with the highest OD₆₀₀. The mutant strain and the wild-type control strain were inoculated into the fermentation medium containing 0, 10, 20, and 40 g/L HA, respectively. Samples were taken at 2 h, 4 h, 6 h, 8 h, 12 h, 16 h, 20 h, 24 h, 36 h, 48 h, and 60 h post-inoculation to measure OD₆₀₀ and the residual glucose content in the fermentation broth. Significant differences in growth rate and glucose consumption rate between the mutant strain and the wild-type strain could be clearly observed.

After being subcultured for 300 generations, the strains in the fermentation broth were streaked for isolation. 100 single-colony strains were selected, inoculated into 5 mL of the fermentation medium containing 40 g/L HA, and cultured at 220 rpm and 30° C. for 20 h. The OD₆₀₀ was measured, and the evolved strain with the highest OD₆₀₀ was designated as viscosity-tolerant Corynebacterium glutamicum CG-HAT of the present invention. The above-described directed evolution effect verification process was then repeated. The directed evolution effect verification of the CG-HAT strain is shown in FIG. 2. With the increase of hyaluronic acid concentration, CG-HAT exhibits higher glucose consumption compared to wild-type Corynebacterium glutamicum ATCC 13032. Therefore, it can be considered that CG-HAT possesses better growth and metabolism ability under high concentrations of hyaluronic acid.

The CG-HAT strain was inoculated into 5 mL of the BHI medium and cultured overnight. The culture was sent to a relevant company for whole-genome sequencing. By comparing with the genome of wild-type Corynebacterium glutamicum ATCC 13032 before evolution used as the reference genome, it is found that multiple gene mutations are occurred in the genome of the viscosity-tolerant Corynebacterium glutamicum strain, with specific mutation information as shown in Table 1.

TABLE 1
Genome mutation information of CG-HAT strain

| | | | | CG- |
|---|---|---|---|---|
| type of gene | genome start | genome termination | reference | HAT |
| mutation | position | position | genome | genome |
| single nucleotide | 76620 | 76620 | G | A |
| variation | | | | |
| single nucleotide | 143559 | 143559 | G | A |
| variation | | | | |
| single nucleotide | 245282 | 245282 | C | A |
| variation | | | | |
| single nucleotide | 286678 | 286678 | G | A |
| variation | | | | |
| single nucleotide | 287836 | 287836 | T | C |
| variation | | | | |
| single nucleotide | 349179 | 349179 | G | A |
| variation | | | | |
| single nucleotide | 547272 | 547272 | G | C |
| variation | | | | |
| single nucleotide | 591573 | 591573 | A | G |
| variation | | | | |
| single nucleotide | 645542 | 645542 | G | A |
| variation | | | | |
| single nucleotide | 661760 | 661760 | T | C |
| variation | | | | |
| single nucleotide | 862902 | 862902 | C | T |
| variation | | | | |
| single nucleotide | 862903 | 862903 | G | A |
| variation | | | | |
| single nucleotide | 862953 | 862953 | C | T |
| variation | | | | |
| single nucleotide | 862961 | 862961 | A | G |
| variation | | | | |
| single nucleotide | 878149 | 878149 | G | A |
| variation | | | | |
| single nucleotide | 933781 | 933781 | A | T |
| variation | | | | |
| single nucleotide | 1136848 | 1136848 | G | T |
| variation | | | | |
| single nucleotide | 1162092 | 1162092 | T | G |
| variation | | | | |
| single nucleotide | 1480319 | 1480319 | A | G |
| variation | | | | |
| single nucleotide | 1589428 | 1589428 | T | C |
| variation | | | | |
| single nucleotide | 1890744 | 1890744 | C | A |
| variation | | | | |
| single nucleotide | 1961649 | 1961649 | C | A |
| variation | | | | |
| single nucleotide | 1961652 | 1961652 | C | T |
| variation | | | | |
| single nucleotide | 1961654 | 1961654 | C | T |
| variation | | | | |
| single nucleotide | 1961657 | 1961657 | T | C |
| variation | | | | |
| single nucleotide | 1961768 | 1961768 | A | T |
| variation | | | | |
| single nucleotide | 1961774 | 1961774 | C | T |
| variation | | | | |
| single nucleotide | 1961785 | 1961785 | T | C |
| variation | | | | |
| single nucleotide | 1961792 | 1961792 | A | T |
| variation | | | | |
| single nucleotide | 1961798 | 1961798 | C | A |
| variation | | | | |
| single nucleotide | 1961807 | 1961807 | G | A |
| variation | | | | |
| single nucleotide | 1961810 | 1961810 | T | A |
| variation | | | | |
| single nucleotide | 1961820 | 1961820 | C | T |
| variation | | | | |
| single nucleotide | 1961836 | 1961836 | C | T |
| variation | | | | |
| single nucleotide | 1962180 | 1962180 | T | A |
| variation | | | | |
| single nucleotide | 1963988 | 1963988 | G | A |
| variation | | | | |
| single nucleotide | 1963993 | 1963993 | T | C |
| variation | | | | |
| single nucleotide | 1964078 | 1964078 | A | C |
| variation | | | | |
| single nucleotide | 1964435 | 1964435 | G | C |
| variation | | | | |
| single nucleotide | 1965170 | 1965170 | A | G |
| variation | | | | |
| single nucleotide | 1965284 | 1965284 | C | T |
| variation | | | | |
| single nucleotide | 1965286 | 1965286 | T | G |
| variation | | | | |
| single nucleotide | 1965326 | 1965326 | A | G |
| variation | | | | |
| single nucleotide | 1965341 | 1965341 | A | T |
| variation | | | | |
| single nucleotide | 1965344 | 1965344 | C | A |
| variation | | | | |
| single nucleotide | 1965349 | 1965349 | C | A |
| variation | | | | |
| single nucleotide | 1965437 | 1965437 | C | G |
| variation | | | | |
| single nucleotide | 1965443 | 1965443 | A | G |
| variation | | | | |
| single nucleotide | 1965445 | 1965445 | C | T |
| variation | | | | |
| single nucleotide | 1965455 | 1965455 | C | T |
| variation | | | | |
| single nucleotide | 1965500 | 1965500 | A | G |
| variation | | | | |
| single nucleotide | 1965503 | 1965503 | T | G |
| variation | | | | |
| single nucleotide | 1965505 | 1965505 | C | T |
| variation | | | | |
| single nucleotide | 1965506 | 1965506 | A | T |
| variation | | | | |
| single nucleotide | 1965514 | 1965514 | A | G |
| variation | | | | |
| single nucleotide | 1965677 | 1965677 | T | G |
| variation | | | | |
| single nucleotide | 2036533 | 2036533 | T | C |
| variation | | | | |
| single nucleotide | 2131457 | 2131457 | A | C |
| variation | | | | |
| single nucleotide | 2391582 | 2391582 | C | T |
| variation | | | | |
| single nucleotide | 2499503 | 2499503 | A | G |
| variation | | | | |
| single nucleotide | 2578510 | 2578510 | T | C |
| variation | | | | |
| single nucleotide | 2587299 | 2587299 | T | G |
| variation | | | | |
| single nucleotide | 2643621 | 2643621 | C | T |
| variation | | | | |
| single nucleotide | 2676334 | 2676334 | G | A |
| variation | | | | |
| single nucleotide | 2676510 | 2676510 | A | G |
| variation | | | | |
| single nucleotide | 2693877 | 2693877 | A | T |
| variation | | | | |
| single nucleotide | 2701401 | 2701401 | T | C |
| variation | | | | |
| single nucleotide | 2749153 | 2749153 | C | A |
| variation | | | | |
| single nucleotide | 2762168 | 2762168 | G | A |
| variation | | | | |
| single nucleotide | 2965869 | 2965869 | C | T |
| variation | | | | |
| single nucleotide | 3026302 | 3026302 | G | C |
| variation | | | | |
| single nucleotide | 3026303 | 3026303 | C | A |
| variation | | | | |
| single nucleotide | 3114280 | 3114280 | G | C |
| variation | | | | |
| single nucleotide | 3118769 | 3118769 | A | G |
| variation | | | | |
| single nucleotide | 3136899 | 3136899 | A | G |
| variation | | | | |
| single nucleotide | 3217551 | 3217551 | A | T |
| variation | | | | |
| single nucleotide | 3272034 | 3272034 | G | A |
| variation | | | | |
| insertion | 76636 | 76636 | — | T |
| insertion | 862572 | 862572 | — | T |
| insertion | 862958 | 862958 | — | CT |
| deletion | 862963 | 862963 | G | — |
| deletion | 1149598 | 1149599 | TC | — |
| insertion | 1904543 | 1904543 | — | T |
| deletion | 2312377 | 2312427 | CGGTGT | — |
| | | | CATCCT | |
| | | | AGAGAT | |
| | | | TAAGGC | |
| | | | TGAAG | |
| | | | AGGATG | |
| | | | ACACC | |
| | | | GTCGAC | |
| | | | GTCGG | |
| insertion | 2774003 | 2774003 | — | GC |
| deletion | 2774005 | 2774006 | GA | — |

(2) Construction of Corynebacterium glutamicum CG-HAT

In order to further determine the key mutation sites affecting the tolerance of the strain, we selected potential key sites 862902, 862903, 862953, 862961, 862958 and 862963 to carry out mutation verification on the genome of wild-type Corynebacterium glutamicum ATCC 13032. Using plasmid pk18mobsacb as a template, primers PK18-F/PK18-R were designed for PCR amplification reaction to obtain a linear vector pk18mobsacb; Corynebacterium glutamicum CG-HAT was taken out of the refrigerator at −80° C. and revived by streaking on a BHI plate. Single colonies were picked and inoculated into 5 mL of an LB medium, and cultured at 220 rpm and 30° C. for 24 h. Genomic DNA was extracted using a cell genome extraction kit. Using the genomic DNA of Corynebacterium glutamicum CG-HAT as a template, primers 862900-F/862900-R were designed to obtain a gene fragment through amplification with a PCR amplification system and procedure. The obtained gene fragment was ligated with the linear vector pk18mobsacb. The above reaction solution was transformed into Escherichia coli Top10. Transformants were selected for plasmid sequencing, and the recombinant plasmid pk18mobsacb-862900 was successfully constructed. The recombinant plasmid pk18mobsacb-862900 above was transformed into wild-type Corynebacterium glutamicum ATCC 13032 using electroshock transformation, and gene-replaced recombinants were screened on plates to obtain Corynebacterium glutamicum CG-HAT-M (the Corynebacterium glutamicum CG-HAT-M is obtained by mutating Corynebacterium glutamicum ATCC 13032, with mutation sites including: mutation of cytosine at site 862902 into thymine; mutation of guanine at site 862903 into adenine; mutation of cytosine at site 862953 into thymine; mutation of adenine at site 862961 into guanine; inserting cytosine and thymine at site 862958; and mutation of guanine at site 862963 by deletion). Corynebacterium glutamicum CG-HAT-M were inoculated into the fermentation medium containing 0, 10, 20, and 40 g/L HA, respectively. Samples were taken post-inoculation to measure OD₆₀₀ and the residual glucose content in the fermentation broth. The results are shown in FIG. 2. The growth rate and glucose consumption rate of the CG-HAT-M strain are close to those of CG-HAT, and significantly faster than those of wild-type Corynebacterium glutamicum ATCC 13032. The results show that the mutations at sites 862902, 862903, 862953, 862961, 862958 and 862963 are the key sites affecting the viscosity tolerance of Corynebacterium glutamicum. These sites are the first 100 bp of the nucleotide sequence of the pyridoxal phosphate transaminase of the pyridoxal phosphate synthesis gene, and multiple mutations have occurred in this sequence. Pyridoxal phosphate participates in nearly 100 kinds of enzyme reactions, including transamination, decarboxylation, side chain cleavage, dehydration, and transsulfuration. These biochemical functions involve multiple metabolic pathways, including the synthesis and catabolism of proteins; gluconeogenesis; the metabolism of UFA; the metabolism of glycogen, sphingomyelin, and steroids; the synthesis of neurotransmitters (serotonin, taurine, dopamine, norepinephrine, and γ-aminobutyric acid); the metabolism of vitamin B6, one-carbon units, vitamin B12, and folate; as well as the synthesis of nucleic acid and DNA. These metabolic pathways are closely associated with the growth of the strain. Therefore, it is hypothesized that the strain may control the synthesis of pyridoxal phosphate to indirectly enhance the utilization of energy substances by cells, and also enhance the metabolic activity of the strain under anaerobic conditions, thus enhancing its anaerobic metabolism ability.

TABLE 2
Genome mutation information of CG-HAT-M strain

| type of | genome | genome | | experimental |
|---|---|---|---|---|
| gene | start | termination | reference | group |
| mutation | position | position | genome | genome |
| single | 862902 | 862902 | C | T |
| nucleotide | | | | |
| variation | | | | |
| single | 862903 | 862903 | G | A |
| nucleotide | | | | |
| variation | | | | |
| single | 862953 | 862953 | C | T |
| nucleotide | | | | |
| variation | | | | |
| single | 862961 | 862961 | A | G |
| nucleotide | | | | |
| variation | | | | |
| insertion | 862958 | 862958 | — | CT |
| deletion | 862963 | 862963 | G | — |

TABLE 3
Primers needed to construct Corynebacterium
glutamicum CG-HAT-M

| primer | | sequence |
|---|---|---|
| name | sequence (5′-3′) | number |
| PK18-F | GAATTCGTAATCATGGTCATAGC | SEQ ID |
| | | NO. 1 |
| PK18-R | AAGCTTGGCACTGGCCGTCG | SEQ ID |
| | AAGCTTGGCACTGGCCGTCG | NO. 2 |
| 862900- | GCTATGACCATGATTACGAATTCTACAC | SEQ ID |
| F | CCTCTCTTTTTTTGTGTTTGTGGGGGTC | NO. 3 |
| | TTGGGCCCCTTGTGCACGTGGCACTCGC | |
| | G | |
| 862900- | CGACGGCCAGTGCCAAGCTTAGACATAA | SEQ ID |
| R | ACTGCTTCGCCTTC | NO. 4 |

### Example 2: Detection of Ability of Corynebacterium glutamicum CG-HAT and Corynebacterium glutamicum CG-HAT-M to Synthesize Hyaluronic Acid

(1) Construction of Recombinant Corynebacterium glutamicum Strain

Gene synthesis was performed based on the hyaluronic acid synthase gene (HasA) derived from Streptococcus zooepidemicus (with the gene sequence of HasA as shown in SEQ ID NO. 20). The synthesized hyaluronic acid synthase gene was subjected to PCR amplification with HasA-F/HasA-R as primers, resulting in a 1000 bp fragment HasA by amplification. Using plasmid pXMJ19 as a template, primers pXMJ-F/pXMJ-R were designed for PCR amplification reaction to obtain a 10000 bp vector pXMJ. The fragment HasA and the vector pXMJ were subjected to enzyme digestion and ligation reactions. The reaction solution was taken, and transformed into E. coli Top10 via the heat-shock method. Transformants were selected for plasmid extraction and sequencing, and the pXMJ-HasA plasmid was successfully constructed.

By utilizing an electroporator, the recombinant plasmid pXMJ-HasA was transformed into viscosity-tolerant Corynebacterium glutamicum CG-HAT and Corynebacterium glutamicum CG-HAT-M screened in Example 1 using a 1 mm electroporation cuvette, with a perforation voltage of 1500 V, a voltage duration of 5 ms, and the electroshock repeated twice. The strains were incubated at 46° C. for 6 min, and then cultured at 220 rpm and 30° C. for 1 h, coated on a BHI plate containing 15 g/L chloramphenicol, and incubated at 30° C. for 48 h. The recombinant strains were named HVCG-HasA and HVCG-HasA-M. Competent cells of HVCG-HasA and HVCG-HasA-M were prepared for subsequent strain construction.

TABLE 4
Primers used in construction of recombinant
plasmid pXMJ-HasA

| primer | | sequence |
|---|---|---|
| name | sequence (5′-3′) | number |
| HasA-F | AAGGAGGCATTTACATGCCTATTTTCAAGAAG | SEQ ID |
| | ACT | NO. 6 |
| HasA-R | GAGCTCGGTACCCGGGGATCCTTATTTAAAAA | SEQ ID |
| | TAGTAACTTTTTTTCTAG | NO. 7 |
| pXMJ-F | GGATCCCCGGGTACCGAGCTC | SEQ ID |
| | | NO. 8 |
| pXMJ-R | GGCATGTAAATGCCTCCTTAAGCTTAATTAAT | SEQ ID |
| | TCTGTTTCCTGT | NO. 9 |

Using plasmid pk18mobsacb as a template, primers PK18-F/PK18-R were designed for PCR amplification reaction to obtain a linear vector PK18; the Corynebacterium glutamicum strains were taken out of the refrigerator at −80° C. and revived by streaking on an LB plate. Single colonies were picked and inoculated into 5 mL of an LB medium, and cultured at 220 rpm and 30° C. for 24 h. Genomic DNA was extracted using a cell genome extraction kit. Using the genomic DNA of Corynebacterium glutamicum as a template, primers ugd-F/ugd-R, glmS-F/glmS-R, and glmM-F/glmM-R were designed to obtain ugd, glmS and glmM genes with a Ptac promoter through amplification with a PCR amplification system and procedure, wherein the Ptac promoter sequence was designed into the ugd-F, glmS-F and glmM-F primers. The ugd, glmS and glmM genes were ligated with the linear vector PK18 in different arrangements and combinations, and introduced into HVCG-HasA to obtain Corynebacterium glutamicum strains containing HasA-ugd, HasA-glmM, HasA-glmS, HasA-ugd-glmM, HasA-ugd-glmS, HasA-glmM-glmS, and HasA-ugd-glmS-glmM, respectively. Using the Corynebacterium glutamicum strain containing HasA only as a control, the yield of hyaluronic acid was determined under the same conditions. The results are as shown in FIG. 4. It is found that the ugd, glmS, and glmM genes can all significantly increase the yield of hyaluronic acid. The Corynebacterium glutamicum strain containing HasA-ugd-glmS-glmM exhibits the highest yield of hyaluronic acid. Therefore, the Corynebacterium glutamicum strain with HasA-ugd-glmS-glmM was selected to increase the yield of hyaluronic acid. The specific construction process is as follows:

The fragments ugd, glmS and glmM and the linear vector PK18 were subjected to enzyme digestion and ligation reactions. The above reaction solution was transformed into Escherichia coli Top10. Transformants were selected for plasmid sequencing, and the recombinant plasmid PK18-Ptac-ugd-glmS-glmM was successfully constructed. The recombinant plasmid above was transformed into HVCG-HasA and HVCG-HasA-M using electroshock transformation to construct recombinant Corynebacterium glutamicum strains HVCG-HasA-Ptac-ugd-glmS-glmM and HVCG-HasA-M-Ptac-ugd-glmS-glmM.

TABLE 5
Primers used in construction of recombinant
plasmid PK18-Ptac-ugd-glmS-glmM

| primer | | sequence |
|---|---|---|
| name | sequence (5′-3′) | number |
| ugd-F | CGACGGCCAGTGCCAAGCTTTTGACAATTAATC | SEQ ID |
| | ATCGGCTCGTATAATGTGTGGTCTCCAGTCCAC | NO. 10 |
| | TCTTCATTGAAAG | |
| ugd-R | GCTATGACCATGATTACGAATTCCTAAAGGTTG | SEQ ID |
| | CGGCCGAGCGCTTC | NO. 11 |
| glmM-F | CGACGGCCAGTGCCAAGCTTTTGACAATTAATC | SEQ ID |
| | ATCGGCTCGTATAATGTGTGGTGTTCAATAGAG | NO. 12 |
| | TTTTGAACAATG | |
| glmM-R | GCTATGACCATGATTACGAATTCTTAGACTTCT | SEQ ID |
| | GCAACCACTG | NO. 13 |
| glmS-F | CGACGGCCAGTGCCAAGCTTTTGACAATTAATC | SEQ ID |
| | ATCGGCTCGTATAATGTGTGGTTATGGTCCTCC | NO. 14 |
| | CAGCTCAGTGT | |
| glmS-R | GCTATGACCATGATTACGAATTCTTATTCGACG | SEQ ID |
| | GTGACAGACTTTGC | NO. 15 |
| Ptac | TTGACAATTAATCATCGGCTCGTATAATGT | SEQ ID |
| | | NO. 16 |

(2) Detection of Yield of Hyaluronic Acid by Recombinant Corynebacterium glutamicum Strain

The yield of a recombinant Corynebacterium glutamicum strain was detected by recombinant Corynebacterium glutamicum strains HVCG-HasA-Ptac-ugd-glmS-glmM and HVCG-HasA-M-Ptac-ugd-glmS-glmM.

250 mL shake flask fermentation production: The recombinant Corynebacterium glutamicum strains HVCG-HasA-Ptac-ugd-glmS-glmM and HVCG-HasA-M-Ptac-ugd-glmS-glmM constructed in Example 2, Step (1) were respectively inoculated into a shaking tube with 5 mL of BHI, and cultured overnight at 220 rpm and 30° C. The seed liquid was transferred into a baffled Erlenmeyer flask with 25 mL of the fermentation medium at an inoculation amount of initial OD₆₀₀=0.2, and cultured at 220 rpm and 30° C. for 3.5 h. Then, IPTG was added at a final concentration of 0.25 mM to induce gene expression, and the fermentation period was 48 h. Wherein, at 20 h and 24 h of fermentation, 2 M NaOH was added to adjust the pH of the fermentation broth to 6.5-7. The fermentation broth was collected and centrifuged at 10000 rpm for 5 min. The supernatant was then subjected to repeated alcohol precipitation twice, followed by determination of the content of hyaluronic acid in the broth using the borax in sulfuric acid-carbazole method. The yields of hyaluronic acid produced by both recombinant Corynebacterium glutamicum strains HVCG-HasA-Ptac-ugd-glmS-glmM and HVCG-HasA-M-Ptac-ugd-glmS-glmM in shake flask fermentation were 10 g/L.

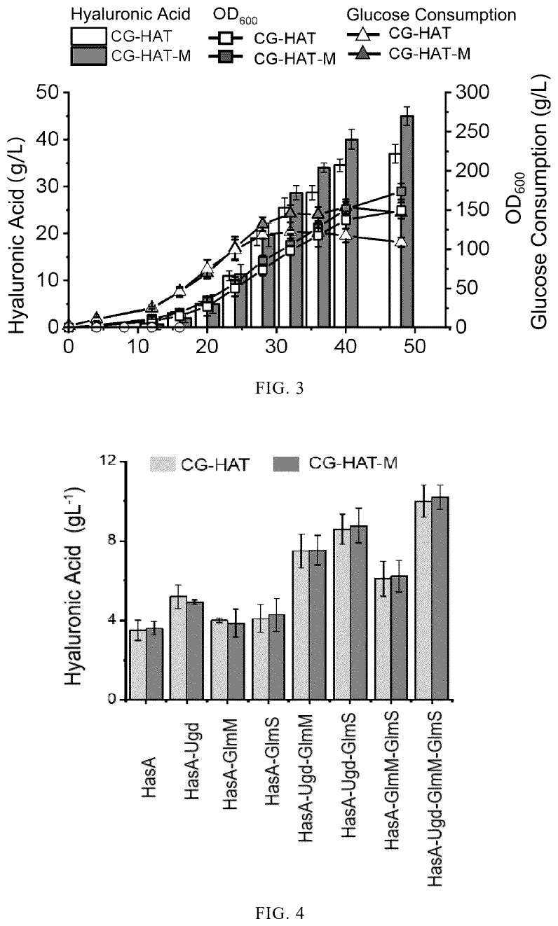

5 L fermentor fermentation production: The recombinant Corynebacterium glutamicum strains HVCG-HasA-Ptac-ugd-glmS-glmM and HVCG-HasA-M-Ptac-ugd-glmS-glmM constructed in Example 2, Step (1) were respectively inoculated into 5 mL of the BHI medium, and cultured overnight at 220 rpm and 30° C. The seed liquid was transferred into a baffled Erlenmeyer flask with 25 mL of the fermentation medium at an inoculation amount of initial OD₆₀₀=0.1, and cultured at 220 rpm and 30° C. for 10 h, and then inoculated into a 5 L fermentor at an inoculation amount of 10%. The initial temperature was set at 30° C., and the rotation speed was 3000 r/min. After 3.5 hours of fermentation, IPTG was added at a final concentration of 0.25 mM to induce gene expression. During the fermentation process, 14% aqueous ammonia was used to control the pH of the fermentation broth at approximately 7, and glucose was fed-batch to maintain its content in the fermenter at approximately 10 g/L. As can be seen from FIG. 3, the final yields of hyaluronic acid by the high viscosity-tolerant recombinant Corynebacterium glutamicum strains HVCG-HasA-Ptac-ugd-glmS-glmM and HVCG-HasA-M-Ptac-ugd-glmS-glmM after fermentation in 5 L fermentor were 44 g/L and 45 g/L, respectively.

### COMPARATIVE EXAMPLE

The recombinant plasmid HasA-ugd-glmS-glmM constructed according to Example 2, Step (1) was transformed into wild-type Corynebacterium glutamicum ATCC 13032, and fermented according to the method of Example 2, Step (2). The results show that the yield of hyaluronic acid fermented by the wild-type Corynebacterium glutamicum strain in shake flask was 6.5 g/L, and in 5 L fermentor was only 32 g/L. The primary reason is that as hyaluronic acid accumulates in the fermentation broth, the wild-type Corynebacterium glutamicum strain cannot carry out normal metabolic activities in the fermentation broth with a high viscosity, thereby affecting hyaluronic acid synthesis.

Obviously, the examples above are only intended to clarify the description, and shall not be construed as limitations to the embodiments. For those of ordinary skill in the art, other changes or variations in different forms can be made on the basis of the above description. It is not necessary and impossible to exhaust all the embodiments here. The obvious changes or variations derived therefrom are still within the scope of protection created by the present invention.

What is claimed is:

1. A viscosity-tolerant Corynebacterium glutamicum strain, wherein the Corynebacterium glutamicum strain is obtained by mutating Corynebacterium glutamicum ATCC 13032, with mutation sites comprising:
(1) mutation of cytosine at site 862902 into thymine;
(2) mutation of guanine at site 862903 into adenine;
(3) mutation of cytosine at site 862953 into thymine;
(4) mutation of adenine at site 862961 into guanine;
(5) inserting cytosine and thymine at site 862958; and
(6) mutation of guanine at site 862963 by deletion.

2. The Corynebacterium glutamicum strain according to claim 1, wherein the mutation sites comprise:

| type of gene | genome start | genome termination | reference | CG-HAT |
|---|---|---|---|---|
| mutation | position | position | genome | genome |
| single nucleotide | 76620 | 76620 | G | A |
| variation | | | | |
| single nucleotide | 143559 | 143559 | G | A |
| variation | | | | |
| single nucleotide | 245282 | 245282 | C | A |
| variation | | | | |
| single nucleotide | 286678 | 286678 | G | A |
| variation | | | | |
| single nucleotide | 287836 | 287836 | T | C |
| variation | | | | |
| single nucleotide | 349179 | 349179 | G | A |
| variation | | | | |
| single nucleotide | 547272 | 547272 | G | C |
| variation | | | | |
| single nucleotide | 591573 | 591573 | A | G |
| variation | | | | |
| single nucleotide | 645542 | 645542 | G | A |
| variation | | | | |
| single nucleotide | 661760 | 661760 | T | C |
| variation | | | | |
| single nucleotide | 862902 | 862902 | C | T |
| variation | | | | |
| single nucleotide | 862903 | 862903 | G | A |
| variation | | | | |
| single nucleotide | 862953 | 862953 | C | T |
| variation | | | | |
| single nucleotide | 862961 | 862961 | A | G |
| variation | | | | |
| single nucleotide | 878149 | 878149 | G | A |
| variation | | | | |
| single nucleotide | 933781 | 933781 | A | T |
| variation | | | | |
| single nucleotide | 1136848 | 1136848 | G | T |
| variation | | | | |
| single nucleotide | 1162092 | 1162092 | T | G |
| variation | | | | |
| single nucleotide | 1480319 | 1480319 | A | G |
| variation | | | | |
| single nucleotide | 1589428 | 1589428 | T | C |
| variation | | | | |
| single nucleotide | 1890744 | 1890744 | C | A |
| variation | | | | |
| single nucleotide | 1961649 | 1961649 | C | A |
| variation | | | | |
| single nucleotide | 1961652 | 1961652 | C | T |
| variation | | | | |
| single nucleotide | 1961654 | 1961654 | C | T |
| variation | | | | |
| single nucleotide | 1961657 | 1961657 | T | C |
| variation | | | | |
| single nucleotide | 1961768 | 1961768 | A | T |
| variation | | | | |
| single nucleotide | 1961774 | 1961774 | C | T |
| variation | | | | |
| single nucleotide | 1961785 | 1961785 | T | C |
| variation | | | | |
| single nucleotide | 1961792 | 1961792 | A | T |
| variation | | | | |
| single nucleotide | 1961798 | 1961798 | C | A |
| variation | | | | |
| single nucleotide | 1961807 | 1961807 | G | A |
| variation | | | | |
| single nucleotide | 1961810 | 1961810 | T | A |
| variation | | | | |
| single nucleotide | 1961820 | 1961820 | C | T |
| variation | | | | |
| single nucleotide | 1961836 | 1961836 | C | T |
| variation | | | | |
| single nucleotide | 1962180 | 1962180 | T | A |
| variation | | | | |
| single nucleotide | 1963988 | 1963988 | G | A |
| variation | | | | |
| single nucleotide | 1963993 | 1963993 | T | C |
| variation | | | | |
| single nucleotide | 1964078 | 1964078 | A | C |
| variation | | | | |
| single nucleotide | 1964435 | 1964435 | G | C |
| variation | | | | |
| single nucleotide | 1965170 | 1965170 | A | G |
| variation | | | | |
| single nucleotide | 1965284 | 1965284 | C | T |
| variation | | | | |
| single nucleotide | 1965286 | 1965286 | T | G |
| variation | | | | |
| single nucleotide | 1965326 | 1965326 | A | G |
| variation | | | | |
| single nucleotide | 1965341 | 1965341 | A | T |
| variation | | | | |
| single nucleotide | 1965344 | 1965344 | C | A |
| variation | | | | |
| single nucleotide | 1965349 | 1965349 | C | A |
| variation | | | | |
| single nucleotide | 1965437 | 1965437 | C | G |
| variation | | | | |
| single nucleotide | 1965443 | 1965443 | A | G |
| variation | | | | |
| single nucleotide | 1965445 | 1965445 | C | T |
| variation | | | | |
| single nucleotide | 1965455 | 1965455 | C | T |
| variation | | | | |
| single nucleotide | 1965500 | 1965500 | A | G |
| variation | | | | |
| single nucleotide | 1965503 | 1965503 | T | G |
| variation | | | | |
| single nucleotide | 1965505 | 1965505 | C | T |
| variation | | | | |
| single nucleotide | 1965506 | 1965506 | A | T |
| variation | | | | |
| single nucleotide | 1965514 | 1965514 | A | G |
| variation | | | | |
| single nucleotide | 1965677 | 1965677 | T | G |
| variation | | | | |
| single nucleotide | 2036533 | 2036533 | T | C |
| variation | | | | |
| single nucleotide | 2131457 | 2131457 | A | C |
| variation | | | | |
| single nucleotide | 2391582 | 2391582 | C | T |
| variation | | | | |
| single nucleotide | 2499503 | 2499503 | A | G |
| variation | | | | |
| single nucleotide | 2578510 | 2578510 | T | C |
| variation | | | | |
| single nucleotide | 2587299 | 2587299 | T | G |
| variation | | | | |
| single nucleotide | 2643621 | 2643621 | C | T |
| variation | | | | |
| single nucleotide | 2676334 | 2676334 | G | A |
| variation | | | | |
| single nucleotide | 2676510 | 2676510 | A | G |
| variation | | | | |
| single nucleotide | 2693877 | 2693877 | A | T |
| variation | | | | |
| single nucleotide | 2701401 | 2701401 | T | C |
| variation | | | | |
| single nucleotide | 2749153 | 2749153 | C | A |
| variation | | | | |
| single nucleotide | 2762168 | 2762168 | G | A |
| variation | | | | |
| single nucleotide | 2965869 | 2965869 | C | T |
| variation | | | | |
| single nucleotide | 3026302 | 3026302 | G | C |
| variation | | | | |
| single nucleotide | 3026303 | 3026303 | C | A |
| variation | | | | |
| single nucleotide | 3114280 | 3114280 | G | C |
| variation | | | | |
| single nucleotide | 3118769 | 3118769 | A | G |
| variation | | | | |
| single nucleotide | 3136899 | 3136899 | A | G |
| variation | | | | |
| single nucleotide | 3217551 | 3217551 | A | T |
| variation | | | | |
| single nucleotide | 3272034 | 3272034 | G | A |
| variation | | | | |
| insertion | 76636 | 76636 | — | T |
| insertion | 862572 | 862572 | — | T |
| insertion | 862958 | 862958 | — | CT |
| deletion | 862963 | 862963 | G | — |
| deletion | 1149598 | 1149599 | TC | — |
| insertion | 1904543 | 1904543 | — | T |
| deletion | 2312377 | 2312427 | CGGTGT | — |
| | | | CATCCT | |
| | | | AGAGAT | |
| | | | TAAGGC | |
| | | | TGAAG | |
| | | | AGGATG | |
| | | | ACACCG | |
| | | | TCGACG | |
| | | | TCGG | |
| insertion | 2774003 | 2774003 | — | GC |
| deletion | 2774005 | 2774006 | GA | - |

3. Use of the Corynebacterium glutamicum strain according to claim 1 in production of mucopolysaccharides.

4. The use according to claim 3, wherein the mucopolysaccharide comprises hyaluronic acid.

5. A recombinant Corynebacterium glutamicum strain, wherein the modification of the recombinant Corynebacterium glutamicum strain comprises: overexpressing a hyaluronic acid synthase, and overexpressing at least one of a glutamine-fructose-6-phosphate aminotransferase, a phosphoglucomutase, and a uridine diphosphate-glucose dehydrogenase in the Corynebacterium glutamicum strain according to claim 1.

6. The recombinant Corynebacterium glutamicum strain according to claim 5, wherein the overexpression is initiated by a Ptac promoter or a Ptrc promoter.

7. A method for producing mucopolysaccharides, comprising a step of fermentation of the recombinant Corynebacterium glutamicum strain according to claim 5.

8. The method according to claim 7, wherein the fermentation comprises inoculating the recombinant Corynebacterium glutamicum strain into a fermentation medium for culture, adding IPTG to induce gene expression, centrifuging the fermentation broth after fermentation, and collecting the supernatant to obtain the mucopolysaccharide.

9. The method according to claim 8, wherein the temperature of the culture is 15-40° C.

10. The method according to claim 8, wherein the pH of the culture is 5-9.


SEQUENCE LISTING (20 sequences)

SEQ ID NO: 1 - DNA, 23 bp, synthetic construct
  source 1..23: mol_type = other DNA
gaattcgtaatcatggtcatagc

SEQ ID NO: 2 - DNA, 20 bp, synthetic construct
  source 1..20: mol_type = other DNA
aagcttggcactggccgtcg

SEQ ID NO: 3 - DNA, 85 bp, synthetic construct
  source 1..85: mol_type = other DNA
gctatgaccatgattacgaattctacaccctctctttttttgtgtttgtgggggtcttgggccccttgtgcacgtggcactcgcg

SEQ ID NO: 4 - DNA, 42 bp, synthetic construct
  source 1..42: mol_type = other DNA
cgacggccagtgccaagcttagacataaactgcttcgccttc

SEQ ID NO: 5 - DNA, 101 bp, synthetic construct
  source 1..101: mol_type = other DNA
actacaccctctctttttttgtgtttgtgggggtcttgggccccttgtgcacgtggcacctgcgcctgggggcaagcgtcgacaagcacaaactttttgct

SEQ ID NO: 6 - DNA, 35 bp, synthetic construct
  source 1..35: mol_type = other DNA
aaggaggcatttacatgcctattttcaagaagact

SEQ ID NO: 7 - DNA, 50 bp, synthetic construct
  source 1..50: mol_type = other DNA
gagctcggtacccggggatccttatttaaaaatagtaactttttttctag

SEQ ID NO: 8 - DNA, 21 bp, synthetic construct
  source 1..21: mol_type = other DNA
ggatccccgggtaccgagctc

SEQ ID NO: 9 - DNA, 44 bp, synthetic construct
  source 1..44: mol_type = other DNA
ggcatgtaaatgcctccttaagcttaattaattctgtttcctgt

SEQ ID NO: 10 - DNA, 79 bp, synthetic construct
  source 1..79: mol_type = other DNA
cgacggccagtgccaagcttttgacaattaatcatcggctcgtataatgtgtggtctccagtccactcttcattgaaag

SEQ ID NO: 11 - DNA, 47 bp, synthetic construct
  source 1..47: mol_type = other DNA
gctatgaccatgattacgaattcctaaaggttgcggccgagcgcttc

SEQ ID NO: 12 - DNA, 78 bp, synthetic construct
  source 1..78: mol_type = other DNA
cgacggccagtgccaagcttttgacaattaatcatcggctcgtataatgtgtggtgttcaatagagttttgaacaatg

SEQ ID NO: 13 - DNA, 43 bp, synthetic construct
  source 1..43: mol_type = other DNA
gctatgaccatgattacgaattcttagacttctgcaaccactg

SEQ ID NO: 14 - DNA, 77 bp, synthetic construct
  source 1..77: mol_type = other DNA
cgacggccagtgccaagcttttgacaattaatcatcggctcgtataatgtgtggttatggtcctcccagctcagtgt

SEQ ID NO: 15 - DNA, 47 bp, synthetic construct
  source 1..47: mol_type = other DNA
gctatgaccatgattacgaattcttattcgacggtgacagactttgc

SEQ ID NO: 16 - DNA, 30 bp, synthetic construct
  source 1..30: mol_type = other DNA
ttgacaattaatcatcggctcgtataatgt

SEQ ID NO: 17 - DNA, 1830 bp, synthetic construct
  source 1..1830: mol_type = other DNA
atgtgtggaattgttggcgcgatcgcgcaacgtgatgtagcagaaatccttcttgaaggtttacgtcgtctggaataccgcggatatgattctgccggtctggccgttgttgatgcagaaggtcatatgacccgcctgcgtcgcctcggtaaagtacagatgctggctcaggcagcggaagaacatcctctgcatggcggcaccggtattgctcacactcgctgggcgacccatggtgaaccttcagaagctaacgcgcatccgcatgtttctgaacacattgtggtggtgcataacggcatcatcgaaaaccatgaaccgctgcgtgaagcgttaaaagcacgtggctataccttcgtttctgaaaccgacaccgaagtgattgcacatttggtgaactgggagctgaaacaaggcggtactctgcgtgaggcagttctgcgtgctatcccgcaactgcgtggtgcgtacggtacagtgatcatggactcccgtcatccggataccctgctggcggcacgttctggtagtccgctggtgattggcctgggtatgggcgaaaactttatcgcttctgaccagctggcgctgttgccagtgactcgtcgttttatcttccttgaagagggcgatattgcggaaatcactcgccgttcggtaaacatcttcgataaaactggcgcggaagtaaaacgtcaggatatcgaatccaatctgcaatatgacgcgggcgataaaggtatttaccgccactacatgcagaaagagatctacgaacagccgaacgcgatcaaaaacacccttaccgggcgcatcagccacggtcaggttgatttaagcgagctgggaccgaacgccgatgaactgctgtcgaaggttgagcatattcagatcctcgcctgtggtacttcttataactccggtatggtttcccgctactggtttgaatcgctggcaggtattccgtgcgacgtcgaaatcgcctctgaattccgctatcgcaaatctgccgtgcgtcgtaacagcctgatgatcaccttgtcacagtctggcgaaaccgcggataccctggcgggcctgcgtctgtcgaaagagctgggttaccttggttcgctggcaatctgtaacgttccgggttcttctctggtgcgcgaatccgatctggcgctgatgaccaacgccggtacggaaattggcgtggcctccaccaaagcattcaccacccagttaactgtgctgttgatgctggtggcgaaactgtctcgcctgaaaggtctggatgcctccattgaacatgacatcgtgcatggtctgcaggcgctgccgagccgtattgagcagatgctgtctcaggacaaacgcattgaagcgctggcagaagatttctctgacaaacatcacgcgctgttcctgggccgtggcgatcagtacccaatcgcgctggaaggtgcattgaagctgaaagagatctcttacattcacgctgaagcctacgctgctggcgaactgaaacacggtccgctggcgctgatagatgccgatatgccggttatcgtcgttgcaccgaacaacgaattgctggaaaaactgaaatccaacattgaagaagttcgcgcgcgtggcggtcagttgtatgtcttcgccgatcaggatgctggttttgtggggagcgataacatgcacatcatcgagatgccgcatgtggaagaggtgattgcaccaatcttctacaccgttccgctgcaactactggcttaccacgttgcgctgatcaaaggcaccgacgttgaccagccgcgtaacctggcaaaatcggtaacggttgagtaa

SEQ ID NO: 18 - DNA, 1338 bp, synthetic construct
  source 1..1338: mol_type = other DNA
atgagtaatcgtaaatatttcggtaccgatgggattcgtggtcgtgtaggggatgcgccgatcacccctgattttgtgcttaagctgggttgggccgcgggtaaagtgctggcgcgccacggctcccgtaagatcatcattggtaaagacacgcgtatttctggctacatgctggagtcagcactggaagcgggtctggcggctgcgggcctttccgcactcttcactggtccgatgccaacaccggccgtggcttatctgacgcgtaccttccgcgcagaggccggaattgtgatatctgcatcgcataacccgttctacgataatggcattaaatttttctctatcgacggcaccaaactaccggatgcagtagaagaggccatcgaagcggaaatggaaaaagagatcagctgtgttgattcggcagaactgggtaaagccagccgtatcgttgatgctgcgggtcgctatatcgagttttgcaaagccacgttcccaaacgaacttagcctcagtgaactgaagattgtggtggattgtgcaaacggtgcgacttatcacatcgcgccgaacgtgctgcgtgaactgggagcgaacgttatcgctatcggttgtgagccgaacggtgtaaacatcaatgccgaagtgggggctaccgacgttcgcgcgctccaggctcgtgtgctggctgaaaaagcggatctcggtattgccttcgacggcgacggcgatcgcgtgattatggttgaccatgaaggcaataaagtcgatggcgatcagatcatgtatatcatcgcgcgtgaaggtcttcgtcagggccagctgcgtggtggcgctgtgggtacattgatgagcaacatggggcttgaactggcgctgaaacagttaggaattccatttgcgcgcgcgaaagtgggtgaccgctacgtactggaaaaaatgcaggagaaaggctggcgtatcggtgcagagaattccggtcatgtgatcctgctggataaaaccactaccggtgacggcatcgttgctggtttgcaggtgctggcggcgatggcgcgtaatcatatgagcctgcacgacctttgcagcggcatgaaaatgttcccgcaaatcctggtaaacgtgcgttacaccgcaggtagcggcgatccgctggagcatgaatcagtcaaagctgtgaccgcagaagttgaagcggcgctgggtagccgtggacgtgtgctgttgcgtaaatccggcaccgaaccgttaattcgcgtgatggtggaaggcgaagacgaagcgcaggtgactgaatttgcacaccgcatcgccgatgcagtaaaagccgtttaa

SEQ ID NO: 19 - DNA, 1167 bp, synthetic construct
  source 1..1167: mol_type = other DNA
atgaaaatcaccatttccggtactggctatgtaggcttgtcaaacgggcttctaatcgcacaaaatcatgaggttgtggcattagatattttaccgtcacgcgttgctatgctgaatgatcggatatctcctattgttgataaggaaattcagcagtttttgcaatcagataaaatacactttaatgccacattagataaaaatgaagcctaccgggatgctgattatgtcatcatcgccactccaaccgactatgatcctaaaactaattatttcaatacatccagtgtagaatcagtaattaaagacgtagttgagataaatccttatgcggttatggtcatcaaatcaacggttcccgttggttttaccgcagcgatgcataagaaatatcgtactgaaaatattattttctctccggaattcctccgcgaaggtaaagccctttacgataatctccatccgtcacgtattgtcatcggtgagcgttcagaacgcgcagaacgttttgctgcgttgttacaggaaggcgcgattaagcaaaatatcccgaccctgtttaccgactccactgaagcagaagcgattaaacttttcgctaatacctatctggcgatgcgcgtagcatactttaatgaactggatagctatgcagaaagtttaggtctgaatactcgccagattatcgaaggcgtttgtctcgatccgcgtattggcaaccattacaacaatccgtcgtttggttatggtggttattgtctgccgaaagataccaagcagttactggcgaactaccagtctgtgccgaataacctgatctcggcaattgtcgatgcaaaccgcacgcgtaaagattttattgccgatgccattttgtcacgcaaaccgcaagtggtgggtatttatcgtctgattatgaagagcgggtcagataacttccgtgcgtcttctattcaggggattatgaagcgtatcaaggcgaaaggcgttgaagtgatcatctacgagccggtgatgaaagaagactcattcttcaactctcgcctggaacgtgatctcgccaccttcaaacaacaagccgacatcattatctccaaccgtatggcagaagagcttaaggatgtggcagataaggtctacacccgcgatctctttggcagcgactaa

SEQ ID NO: 20 - DNA, 1254 bp, synthetic construct
  source 1..1254: mol_type = other DNA
atgagaacattaaaaaacctcataactgttgtggcctttagtattttttgggtactgttgatttacgtcaatgtttatctctttggtgctaaaggaagcttgtcaatttatggctttttgctgatagcttacctattagtcaaaatgtccttatcctttttttacaagccatttaagggaagggctgggcaatataaggttgcagccattattccctcttataacgaagatgctgagtcattgctagagaccttaaaaagtgttcagcagcaaacctatcccctagcagaaatttatgttgttgacgatggaagtgctgatgagacaggtattaagcgcattgaagactatgtgcgtgacactggtgacctatcaagcaatgtcattgttcaccggtcagaaaaaaatcaaggaaagcgtcatgcacaggcctgggcctttgaaagatcagacgctgatgtctttttgaccgttgactcagatacttatatctaccctgatgctttagaggagttgttaaaaacctttaatgacccaactgtttttgctgcgacgggtcaccttaatgtcagaaatagacaaaccaatctcttaacacgcttgacagatattcgctatgataatgcttttggcgttgaacgagctgcccaatccgttacaggtaatattctcgtttgctcaggcccgcttagcgtttacagacgcgaggtggttgttcctaacatagatagatacatcaaccagaccttcctgggtattcctgtaagtatcggtgatgacaggtgcttgaccaactatgcaactgatttaggaaagactgtttatcaatccactgctaaatgtattacagatgttcctgacaagatgtctacttacttgaagcagcaaaaccgctggaacaagtccttctttagagagtccattatttctgttaagaaaatcatgaacaatccttttgtagccctatggaccatacttgaggtgtctatgtttatgatgcttgtttattctgtggtggatttctttgtaggcaatgtcagagaatttgattggctcagggttttggcctttctggtgattatcttcattgttgctctttgtcgtaatattcactatatgcttaagcacccgctgtccttcttgttatctccgttttatggggtactgcatttgtttgtcctacagcccttgaaattgtattctctttttactattagaaatgctgactggggaacacgtaaaaaattattataa